(19) United States
(12) Patent Application Publication
(10) Pub. No.: US 2024/0165141 A1
(43) Pub. Date: May 23, 2024

(54) SYSTEMS AND METHODS FOR TREATMENT OF BACTERIAL VAGINOSIS

(71) Applicant: Daré Bioscience, Inc., San Diego, CA (US)

(72) Inventors: David Friend, San Diego, CA (US); Jennifer Kiang, San Diego, CA (US); Nicolas J. Pacelli, Cedar Park, TX (US); Mark Walters, San Diego, CA (US)

(21) Appl. No.: 18/291,880

(22) PCT Filed: Jul. 25, 2022
(86) PCT No.: PCT/US2022/038201

§ 371 Date: Jan. 24, 2024

Related U.S. Application Data
(60) Provisional application No. 63/331,744, filed on Apr. 15, 2022.
(60) Provisional application No. 63/285,442, filed on Dec. 2, 2021.
(60) Provisional application No. 63/225,872, filed on Jul. 26, 2021.

(51) Int. Cl. A61K 31/7056; A61K 9/00; A61K 9/06; A61K 47/10; A61K 47/36; A61P 15/02

(52) U.S. Cl. CPC A61K 31/7056; A61K 9/0034; A61K 9/06; A61K 47/10; A61K 47/36; A61P 15/02

(57) ABSTRACT

A pharmaceutical composition for use in the treatment of bacterial vaginosis, wherein said composition comprises a poloxamer, a stabilization polymer, and an active ingredient for treating the bacterial vaginosis, preferably clindamycin, wherein said composition has a viscosity of at least 1.5 million mPa·s.

### RELATED APPLICATIONS

This application claims the benefit of priority to U.S. Provisional Application No. 63/225,872, filed Jul. 26, 2021, U.S. Provisional Application No. 63/285,442, filed Dec. 2, 2021, and U.S. Provisional Application No. 63/331,744, filed Apr. 15, 2022, the entire contents of which are incorporated herein by reference in their entireties.

### FIELD

The present disclosure generally relates to the treatment of bacterial vaginosis and other indications.

### BACKGROUND

Bacterial vaginosis (BV) is one of the most common causes of vaginal discharge in women of childbearing age. It is a “dysbiosis” or imbalance in the vaginal microbiota. It may be characterized by a reduction in normally dominant hydrogen peroxide-producing lactobacilli and/or an increase in other organisms, such as anaerobic Gram-negative rod bacteria. BV can lead to a thin white or gray vaginal discharge, and/or an unpleasant “fishy smell” that may be more noticeable after sexual intercourse, or during menses. BV may also contribute to pelvic inflammatory disease, preterm birth, and increased risk of sexually transmitted infections.

### SUMMARY

The present disclosure generally relates to the treatment of bacterial vaginosis and other indications. The subject matter of the present disclosure involves, in some cases, interrelated products, alternative solutions to a particular problem, and/or a plurality of different uses of one or more systems and/or articles.

One aspect as discussed herein is generally drawn to a composition. In one set of embodiments, the composition comprises a poloxamer, a stabilization polymer, and an active ingredient for treating bacterial vaginosis. In some cases, the composition has a viscosity at room temperature of at least 1.5 million cP (centipoise).

The composition, in another set of embodiments, comprises a poloxamer and a stabilization polymer. The composition may have a viscosity at room temperature of at least 1.5 million cP.

In yet another set of embodiments, the composition comprises a poloxamer, a stabilization polymer, and an active ingredient for treating bacterial vaginosis. In some embodiments, the composition is made by a process comprising forming a composition comprising the poloxamer, the stabilization polymer, and the active ingredient, and removing air from the composition.

According to another set of embodiments, the composition comprises a poloxamer, and a stabilization polymer. The composition may be made by a process comprising forming a composition comprising the poloxamer and the stabilization polymer, and removing air from the composition.

In accordance with still another set of embodiments, the composition comprises a poloxamer, a stabilization polymer, and an active ingredient for treating bacterial vaginosis.

The composition, in some embodiments, can be made by a process comprising forming a composition comprising the poloxamer, the stabilization polymer, and the active ingredient, and exposing the composition to a pressure of less than 100 mbar (absolute) for at least 30 minutes.

The composition, in another set of embodiments, comprises a poloxamer and a stabilization polymer. In some embodiments, the composition is made by a process comprising forming a composition comprising the poloxamer and the stabilization polymer, and exposing the composition to a pressure of less than 100 mbar (absolute) for at least 30 minutes.

In yet another set of embodiments, the composition is a composition for treatment of bacterial vaginosis. In one embodiment, at least 90 wt % of the composition consists essentially of a poloxamer, a stabilization polymer, an active ingredient for treating bacterial vaginosis, and water. In another embodiment, at least 90 wt % of the composition consists essentially of a poloxamer, a stabilization polymer, and water.

In one set of embodiments, the composition comprises each of the following: poloxamer 407 at 10-20 wt %; xanthan gum at 1-5 wt %; clindamycin and/or a salt thereof at 1-5 wt %; citrate and/or a citrate salt at 0.5-2 wt %; benzyl alcohol at 0.5-5 wt %; and water at 60-90 wt %. In another set of embodiments, the composition comprises each of the following: poloxamer 407 at 10-20 wt %; xanthan gum at 1-5 wt %; citrate and/or a citrate salt at 0.5-2 wt %; benzyl alcohol at 0.5-5 wt %; and water at 60-90 wt %.

In another set of embodiments, the compostions comprises poloxamer, a stabilization polymer, and clidamycin and/or a salt thereof. In some cases, the composition, when exposed to a vagina of a human subject, releases the clindamycin and/or salt thereof to the vagina over at least 3 days.

Another aspect is generally drawn to a gel for treatment of bacterial vaginosis. In one set of embodiments, the gel comprises each of the following: a poloxamer at 10-20 wt %; a stabilization polymer at 1-5 wt %; and an active ingredient for treating bacterial vaginosis at 1-5 wt %. In another set of embodiments, the gel comprises each of the following: a poloxamer at 10-20 wt %; and a stabilization polymer at 1-5 wt %.

In one set of embodiments, the composition comprises a poloxamer, a stabilization polymer, and clindamycin and/or a salt thereof. In some cases, the composition, when exposed to a vagina of a human subject, releases clindamycin such that after 48 hours or 72 hours, the subject has a blood concentration of at least 0.5 ng/mL, or at least 5 ng/mL of clindamycin.

In another set of embodiments, the composition comprises a poloxamer, a stabilization polymer, and clindamycin and/or a salt thereof. In some embodiments, when 5 g of the composition is immersed in 0.5 ml of saline solution having a pH 4.6, the composition releases sufficient clindamycin into the saline solution to maintain a concentration of at least 1.4 micrograms/ml after 48 hours or 72 hours.

Yet another aspect is generally directed to a method. In one set of embodiments, the method comprises applying, to a vagina of a subject, a composition comprising a poloxamer, a stabilization polymer, and an active ingredient for treating bacterial vaginosis. In some cases, the composition, as applied, has a viscosity of at least 1.5 million cP.

In another set of embodiments, the method comprises applying, to a vagina of a subject, a composition comprising a poloxamer and a stabilization polymer. In certain embodiments, the composition, as applied, has a viscosity of at least 1.5 million cP.

The method, according to another set of embodiments, comprises applying, to the vagina of a subject, a gel having a viscosity at room temperature of at least 1.5 million cP.

According to still another set of embodiments, the method comprises providing a composition comprising a poloxamer, a stabilization polymer, and an active ingredient for treating bacterial vaginosis, and removing air from the composition.

In another set of embodiments, the method comprises providing a composition comprising a poloxamer and a stabilization polymer, and removing air from the composition.

In accordance with yet another set of embodiments, the method comprises providing a composition comprising a poloxamer, a stabilization polymer, and an active ingredient for treating bacterial vaginosis, and exposing the composition to a pressure of less than 100 mbar (absolute) for at least 30 minutes to form a gel.

The method, in still another set of embodiments, comprises providing a composition comprising a poloxamer and a stabilization polymer, and exposing the composition to a pressure of less than 100 mbar (absolute) for at least 30 minutes to form a gel.

The method, in another set of embodiments, is a method of reducing vaginal odor. In one embodiment, the method comprises applying, to a vagina of a subject exhibiting vaginal odor due to bacterial vaginosis, a gel comprising an active ingredient for treating the bacterial vaginosis. In some cases, the gel has a viscosity at room temperature of at least 1.5 million cP.

In another embodiment, the method comprises applying, to a vagina of a subject exhibiting vaginal odor due to bacterial vaginosis, a composition that causes the vaginal odor to decrease, wherein the composition comprises a poloxamer, a stabilization polymer, and an active ingredient for treating the bacterial vaginosis. In some cases, the composition has a viscosity of at least 1.5 million cP when applied to the vagina of the subject.

In still another set of embodiments, the method is a method of reducing vaginal discharge. According to some embodiments, the method comprises reducing discharge from a vagina of a subject by applying, to the vagina of the subject, a gel comprising an active ingredient for treating the bacterial vaginosis. In some cases, the gel has a viscosity at room temperature of at least 1.5 million cP.

In another embodiment, the method comprises reducing discharge from a vagina of a subject by applying, to the vagina of the subject, a composition that causes a decrease of at least 50 vol % in vaginal discharge over a period of at least a week, relative to an equal period before application of the composition. In some cases, the composition comprises a poloxamer, a stabilization polymer, and an active ingredient for treating bacterial vaginosis. In certain embodiments, the composition has a viscosity of at least 1.5 million cP when applied to the vagina of the subject.

The method, in yet another set of embodiments, is a method of treating recurrent bacterial vaginosis. In certain embodiments, the method comprises treating a subject having recurrent bacterial vaginosis by applying, to a vagina of a subject, a gel comprising an active ingredient for treating the bacterial vaginosis. In some cases, the gel has a viscosity at room temperature of at least 1.5 million cP.

The method, in another embodiment, comprises treating a subject having recurrent bacterial vaginosis by applying, to a vagina of a subject, a composition comprising a poloxamer, a stabilization polymer, and an active ingredient for treating the bacterial vaginosis. In some cases, the composition has a viscosity of at least 1.5 million cP when applied to the vagina of the subject. In some embodiments, the recurrent bacterial vaginosis is identified by at least 3 separate diagnoses of bacterial vaginosis within a 365-day period within the subject.

In another embodiment, the method comprises applying, to a vagina of a subject, a composition comprising a poloxamer, a stabilization polymer, and clindamycin and/or a salt thereof. In some embodiments, the composition releases the clindamycin and/or salt thereof at a rate such that the vagina has a mucosal concentration of the clindamycin and/or salt thereof of at least 100 micrograms/g.

In one embodiment, the method comprises applying, to a vagina of a subject, a composition comprising a poloxamer, a stabilization polymer, and an active ingredient for treating bacterial vaginosis, wherein the composition, as applied, has a viscosity such that the composition releases the active ingredient for at least 3 days into the vagina of the subject.

In another embodiment, the method comprises applying, to a vagina of a subject, a composition comprising a poloxamer, a stabilization polymer, and clidamycin and/or a salt thereof, wherein the composition releases the clidamycin and/or salt thereof into the vagina such that, 3 days after application of the composition, the vagina has at least 2 ppm by weight of clindamycin.

The method, in yet another set of embodiments, comprises applying, to a vagina of a subject, a composition comprising a poloxamer, a stabilization polymer, and clidamycin and/or a salt thereof, wherein the composition releases the clidamycin and/or salt thereof into the vagina such that, 3 days after application of the composition, the subject has a plasma concentration of clindamycin of at least 0.5 ng/ml.

According to still another set of embodiments, the method comprises applying, to a vagina of a subject, a composition comprising a poloxamer, a stabilization polymer, and an active ingredient for treating bacterial vaginosis, the active ingredient having a half-life in the plasma of the subject, wherein the composition releases the active ingredient into the vagina of the subject over at least 18 half-lives.

In one set of embodiments, the method is a method for reducing leakage of a composition from the vagina of a subject. In some cases, the method comprises applying, to a vagina of a subject, a composition comprising a poloxamer, a stabilization polymer, and an active ingredient for treating bacterial vaginosis. In certain embodiments, the composition, as applied, has a viscosity of at least 1.5 million cP.

Several methods are disclosed herein of administering a subject with a compound for prevention or treatment of a particular condition. It is to be understood that in each such aspect of the disclosure, the disclosure specifically includes, also, the compound for use in the treatment or prevention of that particular condition, as well as use of the compound for the manufacture of a medicament for the treatment or prevention of that particular condition.

In another aspect, the present disclosure encompasses methods of making one or more of the embodiments described herein, for example, a composition for the treatment of bacterial vaginosis and other indications. In still another aspect, the present disclosure encompasses methods of using one or more of the embodiments described herein, for example, a composition for the treatment of bacterial vaginosis and other indications.

In an embodiments, provided herein are kits comprising the compositions and gels described herein. For example, the kit includes an applicator suitable for vaginal application.

In other examples, the applicator is pre-filled with the compositions or the gels described herein. In other embodiments, the applicator is not-prefilled with the compositions or the gels described herein. In some embodiments, the compositions or gels described herein are at refrigerated temperatures (e.g., about 4° C.) prior to pre-filling the applicator.

In some embodiments, the kit further includes one or more instructions for inserting the applicator into the vagina. In some embodiments, the kit further includes one or more instructions for filling the applicator with the composition or gel described herein. In additional embodiments, the kit includes instructions for decreasing vaginal odor, reducing vaginal discharge, treating bacterial vaginosis, treating recurring bacterial vaginosis by applying the applicator filled with the compositions or gels described herein.

Other advantages and novel features of the present disclosure will become apparent from the following detailed description of various non-limiting embodiments of the disclosure when considered in conjunction with the accompanying figures.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 illustrates the viscosity of a composition as a function of temperature, in accordance with one embodiment as described herein;

FIG. 2 illustrates a decrease in vaginal odor after treatment, relative to a placebo, in accordance with another embodiment;

FIG. 3 illustrates decrease in vaginal discharge after treatment, relative to a placebo, in accordance with yet another embodiment;

FIG. 4 illustrates clindamycin availability in the vagina after treatment, in still another embodiment;

FIG. 5 illustrates clindamycin availability in blood plasma after treatment, in still another embodiment;

FIG. 6 is a table illustrating plasma clindamycin concentrations, in one embodiment; and

FIG. 7 is a table illustrating vaginal clindamycin concentrations, in another embodiment.

### DETAILED DESCRIPTION

The present disclosure generally relates to the treatment of bacterial vaginosis and other indications. In some cases, such treatments may be beneficial for subjects with recurrent bacterial vaginosis. The composition may decrease vaginal odor and/or vaginal discharge, which may be due to a decrease in the bacteria causing vaginosis. In some embodiments, a composition such as a gel may be applied to the vagina of a subject that is relatively viscous, for example, with a viscosity at room temperature of at least 1.5 million cP. This may be advantageous, in certain embodiments, for reducing leakage or leakage-associated problems from the vagina after application. The composition may also contain an active ingredient, e.g., for treating bacterial vaginosis, such as clindamycin and/or a salt thereof. Compositions having such relatively high viscosities may be useful, for example, to prevent the composition from readily exiting the vagina or degrading too quickly. This may, for example, allow the composition to release the active ingredient over a relatively long period of time to the vagina. In some embodiments, such compositions may be prepared by removing air from the composition to increase its viscosity or cause the composition to form a gel, etc. In addition, certain embodiments as described herein are generally directed to techniques for making or using such compositions, kits including such compositions, or the like.

One aspect as discussed herein is generally directed to compositions having relatively high viscosities, or resistance to flow or deformation. In some embodiments, such compositions may have viscosities of, for example, at least 1 million cP, 3 million cP, or more, or other viscosities as discussed in more detail below. Fluids with such relatively high viscosities do not flow easily and may even resist flow due to gravity in some cases. As mentioned, this may be useful, in certain embodiments, for reducing leakage from the vagina after application of the composition.

The compositions may contain a polymer, which can increase the viscosity of the composition. For example, the polymer may include a poloxamer, which may form a gel in some cases. In addition, in some cases, the composition may also contain xanthan gum, e.g., that can also act as a stabilizer or a thickening agent, which may help increase viscosity. The composition may also contain an active ingredient, such as clindamycin, which can be released from the composition over a suitable period of time. The composition may be applied to the vagina or another suitable body cavity of a subject, for example, where release of the active ingredient is desired, e.g., to treat or prevent an indication, such as bacterial vaginosis. For example, the subject may be a female having or at risk for bacterial vaginosis, or another indication. More details of these and other compositions, in accordance with various embodiments, are provided below.

Such compositions containing relatively high viscosities may be particularly useful, in certain embodiments, to prevent or slow the composition from readily exiting or leaking from the vagina (or other cavity), and/or from degrading too quickly after application. This may be used, for example, to assist the delivery of an active ingredient to the subject, e.g., to the vagina of the subject. For instance, fluids with relatively high viscosities may release the active ingredient more slowly and/or uniformly, thereby allowing the active ingredient to be delivered to the subject over a longer period of time. Higher viscosities may also better resist the natural function of the vagina to discharge. Thus, as discussed in more detail herein, the active ingredient may be delivered, e.g., at suitably effective concentrations or amounts, for example, over a period of at least a day, a week, or even longer in some cases. In addition, in some embodiments, only a single dose of the composition may be required to treat a subject, e.g., since the composition does not readily exit the vagina.

Although other techniques for delivering active ingredients to the vagina (or other body cavity) may also involve the use of relatively high viscosities fluids, these fluids are often selected to have lower viscosities at room temperatures (e.g., about 25° C.) and higher viscosities at body temperatures (e.g., 37° C.). For example, they may be a liquid at room temperature, but become a gel at body temperature. In addition, they may not have viscosities as high as 1 million cP or more; as an example, such a composition may have a viscosity of 300,000 cP at room temperature (and be relatively flowable), increasing to only about 800,000 cP at body temperature.

In contrast, the compositions as discussed herein may have relatively high viscosities, for instance, viscosities as high as 1 million cP or more, even at room temperature. It should be noted that such compositions, due to their high viscosities, are actually difficult to manufacture; accordingly, most other techniques will use compositions with either lower viscosities, or viscosities that are at least low at room temperatures, typically well below 1 million cP. However, without wishing to be bound by any theory, it is believed that having a high initial viscosity, e.g., upon application to the vagina, may be surprisingly useful for rapid symptom relief. A composition with a relatively high viscosity is less able to lose the active ingredient, i.e., prior to application to the vagina, and thus retains the active ingredient for release to the subject more readily than a liquid or less viscous composition. For example, the composition may at least partially seal the vagina better, e.g., to promote treatment with the active ingredient. In addition, a composition with a relatively high viscosity may itself also stay within the vagina longer, e.g., such that the composition would be less able to leak out of the vagina after application. For example, in some embodiments, no more than 25 wt %, no more than 20 wt %, no more than 15 wt %, no more than 10 wt %, no more than 5 wt %, no more than 2 wt %, or no more than 1 wt % of the composition may leak out of the vagina after application.

Furthermore, in some embodiments, the presence of the gel itself may be beneficial, i.e., even without the presence of an active ingredient such as clindamycin. Thus, it should be understood that an active ingredient is not always required. Accordingly, such compositions may produce better symptom resolution, e.g., as compared to formulations with relatively low viscosities at room temperature.

Such high viscosities can be achieved, in various embodiments, using techniques such as removing air from the composition, which may increase its viscosity and/or cause it to form a gel. Other techniques may also be used, including any of those described herein. For example, in certain embodiments, a composition may be prepared, e.g., comprising poloxamer, an active ingredient such as clindamycin, xanthan gum, water, etc., and the composition treated to remove air from the composition, for example, to reduce the composition to 15 vol % air, or less. Techniques for removing air include, but are not limited to, a variety of techniques, such as centrifugation or exposure to relatively high vacuums, e.g., less than 100 mbar. In addition, it should be understood that the composition need not be limited to those described above, and in other embodiments, other compositions may also be used. Examples of such compositions follow.

In addition, in accordance with certain embodiments, compositions as discussed herein may have relatively high viscosities such as those described herein may be particularly useful to treat subjects having bacterial vaginosis, or other indications. In some embodiments, the bacterial vaginosis may be determined by at least 3 separate diagnoses of bacterial vaginosis within a 365-day period within a subject. Without wishing to be bound by any theory, it is believed that such compositions may be able to prevent the composition from readily exiting the vagina, which may facilitate treatment of bacteria, e.g., that causes bacterial vaginosis, including recurrent bacterial vaginosis. This may result in improved subject outcomes, and in some cases, over a relatively short period of time (e.g., within 1 or 2 days, or within 3 days, etc.). For instance, such subjects may exhibit reduced vaginal discharge, reduced vaginal odors, increased vaginal pH, lower clue cell counts (which may indicate a smaller bacterial load), lower Nugent scores (which may indicate the degree of bacterial vaginosis), or the like, e.g., as discussed herein.

For example, in one set of embodiments as discussed herein is generally directed to systems and methods of reducing vaginal discharge. In some cases, subjects having bacterial vaginosis exhibit relatively large amounts of vaginal discharge. While healthy vaginas exhibit some amounts of discharge, those having bacterial vaginosis often exhibit significantly elevated vaginal discharge. In some cases, the discharge may exhibit a change in color, consistency, odor, etc., as compared to normal or healthy vaginal discharge. However, in certain embodiments, a vagina treated as discussed herein may exhibit a significantly less amount of vaginal discharge, e.g., as compared to before treatment. In addition, in some embodiments, there may be a significantly less amount of vaginal discharge that can be determined 1 day after treatment, 2 days after treatment, 3 days after treatment, etc.

Also, in certain embodiments, systems and methods as described herein may be used to decrease or reduce vaginal odor. Although vaginal discharge has a relatively mild odor, bacterial vaginosis is often associated with a stronger, unpleasant odor, which is sometimes described as a “fishy” smell associated with the discharge. However, in some embodiments, a vagina treated as discussed herein may exhibit a decrease in odor after treatment, e.g., as compared to before treatment. In addition, in some embodiments, there may be a significantly less amount of odor that can be determined 1 day after treatment, 2 days after treatment, 3 days after treatment, etc.

Without wishing to be bound by any theory, it is believed that such decreases in odor and/or discharge may be caused by action of composition such as discussed herein on bacteria within the vagina, e.g., that cause bacterial vaginosis. In some embodiments, improvements to the vagina may be determinable as improved vaginal function. For example, improved vaginal function may be determinable as an improvement to vaginal pH. Normal pH of the vagina is around 3.8 to 4.5, but in bacterial vaginosis, the pH may be elevated (less acidic), which is more favorable to bacterial infections. Accordingly, in certain embodiments, a vagina treated as discussed herein may exhibit improved functionality, i.e., as evidenced by a more acidic environment and a decrease in pH to less than 4.5. In addition, in some embodiments, such pH changes may be relatively rapid, e.g., determinable 1 day after treatment, 2 days after treatment, 3 days after treatment, etc.

In another embodiment, improved vaginal function may be determined by determining a decrease in clue cells, which are epithelial cells of the vagina that may exhibit a distinctive stippled appearance when being covered or infected with bacteria. Improved vaginal function and reduced bacterial infection can thus be determined as a decrease in clue cells after treatment, as compared to before treatment. For example, a vagina that is treated as discussed herein may exhibit improved functionality, i.e., as evidenced by a decrease in clue cells. In addition, in some embodiments, such changes in clue cells may be relatively rapid, e.g., determinable 1 day after treatment, 2 days after treatment, 3 days after treatment, etc.

In yet another embodiment, the composition, when delivered to the vagina, may be able to release sufficient amounts of an active ingredient, such as clindamycin, into the vagina such that the active ingredient can be taken systematically into the blood of the subject. In some cases, detectable amounts of the active ingredient (such as clindamycin) may be present in the vagina of the subject after relatively long periods of time, e.g., after 24 hours, 48 hours, or 72 hours. For instance, after such times, the subject may have at least 1 ppm, at least 2 ppm, at least 3 ppm, at least 5 ppm, at least 10 ppm, at least 20 ppm, at least 30 ppm, at least 50 ppm, at least 100 ppm, at least 200 ppm, at least 300 ppm, at least 500 ppm, or at least 1,000 ppm (by weight, e.g., micrograms per gram of vagina mucus). In addition, in some cases, detectable amounts of the active ingredient may be present in the blood after relatively long periods of time, e.g., after 24 hours, 48 hours, or 72 hours. For instance, after such times, the subject may have at least 0.5 ng/ml, at least 1 ng/ml, at least 3 ng/ml, at least 5 ng/ml, at least 10 ng/ml, at least 30 ng/ml, at least 50 ng/ml, or at least 100 ng/ml of an active ingredient, such as clindamycin, in her bloodstream. In addition, in some cases, the amount of release of the active ingredient by the composition can be determined using relatively simple in vitro tests. For example, in some cases, the composition may have a viscosity such that, when 5 g or 5 ml of the composition is immersed in 0.5 ml of saline solution having a pH 4.6, releases sufficient active ingredient (e.g., clindamycin) to maintain a concentration of 1.4 micrograms/ml or 1.4 micrograms/g after relatively long periods of time, e.g., after 24 hours, 48 hours, or 72 hours.

In some aspects, a composition may include one or more poloxamers, xanthan gum, and/or another stabilization polymer, and an active ingredient such as clindamycin or a pharmaceutically acceptable salt thereof. Water may be present in some cases, e.g., such that the composition is a gel, and/or has a relatively high viscosity at room temperature, such as is described herein. Other components may be present as well in certain embodiments, for instance, citrate and/or a citrate salt, benzyl alcohol, or the like. These may act, for example, as excipients, preservatives, antimicrobials, bulking agents, stabilizers, antioxidants, buffers, pH regulating agents, or the like. In addition, in some cases, other components that increase the viscosity of the composition may also be used, for example, hyaluronic acid, alginic acid, modified celluloses such as hydroxypropyl methylcellulose, in addition or instead of poloxomer.

As mentioned, the composition may include one or more poloxamers in one set of embodiments. The poloxamer may be used to increase the viscosity of the composition, e.g., as described herein. In some cases, sufficient poloxamer may be present to cause the composition to form a gel, e.g., at room temperature (about 25° C.) and/or body temperature (about 37° C.). Furthermore, in some cases, the composition may have a gelling temperature, but the gel temperature may be in a range that is physiologically irrelevant. For instance, the gelling temperature may be above 40° C., or below 25° C., and thus, the composition does not change phase or gel at normal physiological or body temperatures.

Poloxamers generally include any of a variety of polyoxyethylene-polyoxypropylene triblock copolymers. In some cases, the poloxamer may be a nonionic block copolymer composed of a central hydrophobic chain of polyoxypropylene (polypropylene oxide) flanked by two hydrophilic chains of polyoxyethylene (polyethylene oxide). In some embodiments, the poloxamers may be soluble in water and other polar and non-polar solvents.

Because the lengths of the polymer blocks can be independently customized, many different poloxamers exist that have slightly different properties. For example, the polxoxamer may have a structure:

HO—[CH₂—CH₂—O]ₐ—[CH₂—CH(CH₃)—O]_{b}—[CH₂—CH₂—O]ₐ—H.

The structure includes a core of propylene oxide (represented by “b” in the above figure), flanked by ethylene oxide subunits (represented by “a” in the above figure), typically on both sides. The sum of the two a's may be, for example, from 50 to 500, from 100 to 300, from 150 to 250, or 200. As another example, a may be between 99 and 103, e.g., 101. b may be, for example, from 30 to 100, from 50 to 80, from 60 to 70, or 65. As another example, b may be between 54 and 58, e.g., 56.

In some embodiments, the ethylene oxide subunits forming the poloxamer may be in molar excess to the propylene oxide subunits. For example, in certain embodiments, the ratio of ethylene oxide subunits to propylene oxide subunits (i.e., a:b) may be, for example, from 3:1 to 5:1, or from 2:1 to 4:1.

Several suitable poloxamers can be readily obtained commercially, including poloxamer 407, Pluronic® F-127, or the like. The composition may include a single poloxamer, or more than one type of poloxamer. In some cases, at least 50 wt %, at least 60 wt %, at least 70 wt %, at least 80 wt %, or at least 90 wt % of the poloxamer within the composition is a single type of poloxamer, for example, poloxamer 407 or Pluronic® F-127.

The molecular weight of the poloxamer may be, in one embodiment, from 5 kDa to 25 kDa. In some instances, the molecular weight of the copolymer may be from 9 kDa to 16 kDa. In some cases, the molecular weight of the poloxamer may be at least 1 kDa, at least 2 kDa, at least 3 kDa, at least 4 kDa, at least 5 kDa, at least 7 kDa, at least 9 kDa, at least 10 kDa, at least 15 kDa, at least 16 kDa, at least 20 kDa, at least 25 kDa, at least 50 kDa, etc. In addition, in certain embodiments, the molecular weight of the poloxamer may be no more than 50 kDa, no more than 25 kDa, no more than 20 kDa, no more than 16 kDa, no more than 15 kDa, no more than 10 kDa, no more than 9 kDa, no more than 5 kDa, no more than 4 kDa, no more than 3 kDa, no more than 2 kDa, no more than 1 kDa, etc. Combinations of any of these are also possible. For instance, the poloxamer may have a molecular weight of between 10 kDa and 15 kDa. As other non-limiting examples, the molecular weight may be between 3 kDa and 5 kDa, between 2 kDa and 4 kDa, between 5 kDa and 20 kDa, between 9 kDa and 16 kDa, etc. The molecular weight, in some cases, may be determined as a weight average molecular weight.

In certain embodiments, the poloxamer may be present within the composition at concentrations of at least 1 wt %, at least 2 wt %, at least 3 wt %, at least 5 wt %, at least 7 wt %, at least 10 wt %, at least 15 wt %, at least 20 wt %, at least 25 wt %, at least 30 wt %, at least 40 wt %, or at least 50 wt %. In addition, in some embodiments, the poloxamer may be present within the composition at concentrations of no more than 50 wt %, no more than 40 wt %, no more than 30 wt %, no more than 25 wt %, no more than 20 wt %, no more than 15 wt %, no more than 10 wt %, no more than 7 wt %, no more than 5 wt %, no more than 3 wt %, no more than 2 wt %, etc. Combinations of any of these are also possible in other embodiments. For example, the poloxamer may be present in a composition at between 10 wt % and 20 wt %, between 5 wt % and 15 wt %, between 15 wt % and 30 wt %, etc.

In one embodiment, the poloxamer used in the composition is Pluronic® F-127. In Pluronic® F-127, the sum of two a's in the above block polymer structure may be 200, and b may have a value of 65. In Pluronic® F-127, the ratio of the sum of two a's to b in the poloxamer (i.e., a:b) may be from 2:1 to 4:1. Tables 1 and 2 illustrate chemical composition and specifications of Pluronic® F-127.

TABLE 1
Chemical Composition of Pluronic ® F-127

| | | | | | | Number average | | |
|---|---|---|---|---|---|---|---|---|
| | | | | Content of | | molecular | | Number of |
| | | | | Oxyethylene | Molecular | weight | Polydispersity | repeating |
| Pluronic | Poloxamer | a | b | (Percent) | Weight | (g/mol) | index | units |
| F 127 NF | 407 | 101 | 56 | 71.5-74.9 | 9840-14600 | 10000 | 1.2 | EG₂₀₀PG₆₅PH₀ |

TABLE 2
Specifications of Pluronic ® F-127

| | pH | Cloud | | | | | Ethylene | Propylene | 1,4 |
|---|---|---|---|---|---|---|---|---|---|
| Physical | (2.5% | point, | APHA | | BHT, | Unsaturation | Oxide, | Oxide, | dioxane, |
| Form | in water) | 10% | color | % H₂O | ppm | mEq/g | ppm | ppm | ppm |
| Solid | 5.0-7.5 | >100° C. | 120 | Cast solid 0.4 max. | 50-125 | 0.048 ± 0.017 | 1 max. | 5 max. | 0.002% |
| | | | max. | Prill 0.75 max. | | | | | max. |

In addition, in one set of embodiments, the composition may comprise xanthan gum, and/or another stabilization polymer. Examples of other stabilization polymers include hyaluronic acid, alginic acid, modified celluloses such as hydroxypropyl methylcellulose, or others such as described herein. Xanthan gum generally refers to a high molecular weight polysaccharide used as a food additive and rheology modifier, as would be known by those of ordinary skill in the art. In addition, many such xanthan gums are readily available commercially. Xanthan gum may be produced, as a non-limiting example, by a process involving fermentation of glucose or sucrose by the Xanthomonas campestris bacterium. In some embodiments, the backbone of the polysaccharide chain may have two beta-D-glucose units linked through the 1 and 4 positions. The side chains are formed of two mannose and one glucuronic acid, so the chain has repeating modules of five sugar units. The side chain is linked to every other glucose of the backbone at the 3 position. About half of the terminal mannose units have a pyruvic acid group linked as a ketal to its 4 and 6 positions. The other mannose unit has an acetyl group at the 6 positions. Two of these chains may be aligned to form a double helix, giving a rather rigid rod configuration that accounts for its high efficiency as a viscosifier of water.

However, it should be understood that not all xanthan gums have precisely the above molecular configuration or properties, and that xanthan gums may vary in molecular composition, e.g., depending on the source of the xanthan gum, especially those arising from different biological sources. In addition, other stabilization polymers instead of (or in addition to) xanthan gum can be used, for example, KELTROL® BT and/or KELTROL® RD, KELZAN® XC, KELZAN® XCD, KELZAN® D, KELZAN® CC, XANTURAL® 180, XANTURAL® 75, or the like, all of which can be obtained commercially from various suppliers.

The molecular weight of the xanthan gum or other stabilization polymer can vary. For instance, the xanthan gum or other stabilization polymer may have any suitable molecular weight, for example, at least about 1 million, at least about 2 million, at least about 5 million, at least about 10 million, at least about 25 million, or at least about 50 million. In other embodiments, the molecular weight can vary from about one million to 50 million, e.g., depending upon various factors such as how it is prepared. In some embodiments, the molecular weight can range from approximately 1 million to approximately 25 million, e.g., as measured by a Brookfield Viscometer or other suitable device. In yet other embodiments, the molecular weight may be, for example, 1, 2, 3, 4, or 5 (+/−0.5) million, or 6, 7, 8, 9, 10, 11, 12, 13, 14, 15, 16, 17, 18, 19, 19, 20, 21, 22, 23, 24, or 25 (+/−2) million. Still other molecular weights are also possible.

The xanthan gum (and/or another stabilization polymer) may be present within the composition at concentrations of at least 0.1 wt %, at least 0.2 wt %, at least 0.3 wt %, at least 0.4 wt %, at least 0.5 wt %, at least 0.7 wt %, at least 1 wt %, at least 1.5 wt %, at least 2 wt %, at least 2.5 wt %, at least 3 wt %, at least 3.5 wt %, at least 4 wt %, at least 4.5 wt %, at least 5 wt %, at least 5.5 wt %, at least 6 wt %, at least 6.5 wt %, at least 7 wt %, at least 7.5 wt %, at least 8 wt %, at least 8.5 wt %, at least 9 wt %, at least 9.5 wt %, at least 10 wt %, etc. In addition, in some cases, the xanthan gum and/or other stabilization polymer may be present at no more than 10 wt %, no more than 9.5 wt %, no more than 9 wt %, no more than 8.5 wt %, no more than 8 wt %, no more than 7.5 wt %, no more than 7 wt %, no more than 6.5 wt %, no more than 6 wt %, no more than 5.5 wt %, no more than 5 wt %, no more than 4.5 wt %, no more than 4 wt %, no more than 3.5 wt %, no more than 3 wt %, no more than 2.5 wt %, no more than 2 wt %, no more than 1.5 wt %, no more than 1 wt %, no more than 0.8 wt %, no more than 0.6 wt %, no more than 0.4 wt %, no more than 0.2 wt %, etc. In addition, in certain instances, combinations of any of these ranges are also possible. For example, the xanthan gum and/or other stabilization polymer may be present at between 1 wt % and 5 wt %, between 0.5 wt % and 2 wt %, between 0.5 wt % and 5 wt %, between 0.5 wt % and 2 wt %, or the like.

In one set of embodiments, the composition may also comprise an active ingredient. The active ingredient may be one suitable for treating bacterial vaginosis, or another indication. For instance, the active ingredient may be one suitable for treatment of a subject for a condition when the active ingredient is delivered to the vagina, or another suitable body cavity. In some embodiments, the active ingredient is present in a therapeutically effective amount. One or more than one active ingredient may be used, depending on the embodiment.

For example, in certain embodiments, the active ingredient may include clindamycin or a salt thereof (e.g., clindamycin phosphate, clindamycin HCl, etc.), wherein the weight of the clindamycin is based on the weight of the free base. Other suitable active ingredients include, but are not limited to, lincomycin, metronidazole, clotrimazole, secnidazole, ornidazole, tinidazole, probiotics, boric acid, etc., and/or pharmaceutically acceptable salts thereof. The pharmaceutical formulation may also include an antibiotic as an active ingredient in certain cases.

Additional non-limiting examples of suitable active ingredients may also found in a patent application filed on even date herewith, entitled “Systems and Methods for Treatment of Bacterial Vaginosis and Other Indications,” by Friend et al; and in U.S. Ser. No. 63/225,872, filed Jul. 26, 2021, entitled “Systems and Methods for Treatment of Bacterial Vaginosis and Other Indications,” by Friend et al.

If a salt is present, the salt may be a pharmaceutically acceptable salt in some embodiments. Pharmaceutically acceptable salts include salts which are, within the scope of sound medical judgment, suitable for use in contact with the tissues of a subject (e.g., a human) without undue toxicity, irritation, allergic response, and the like, and are commensurate with a reasonable benefit/risk ratio. Pharmaceutically acceptable salts may also be salts that are generally safe, non-toxic and neither biologically nor otherwise undesirable and includes that which is acceptable for human pharmaceutical use. Pharmaceutically acceptable salts are well known in the art. Pharmaceutically acceptable salts of the compounds describe herein include those derived from suitable inorganic and organic acids and bases. Examples of pharmaceutically acceptable, nontoxic acid salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, quaternary salts.

In some cases, the active ingredient may be released from the composition over any suitable period of time. For example, a therapeutically effective amount of the active ingredient may be released from 1 to 21 days. In some instances, a therapeutically effective amount of the active ingredient may be released up to about 1 to 7 days, about 5 or 15 days, or about 8 to 29 days following administration. As still other examples, the active ingredient may be released at therapeutically effective amounts from the composition, following application to the subject, for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 or more days. In some embodiments, only a single application of the composition to the subject may be needed. However, in other embodiments, the composition may be applied to the subject more than once, for example, in applications separated by any of the lengths of time discussed herein.

For example, in some cases, tₘₐₓ, or the time at which the maximum concentration of the active ingredient is present in the vagina (due to release by the composition) may be at least 1 day, at least 2 days, at least 3 days, etc., or other times such as those described above. In addition, in some embodiments, tmax may be less than 30, 29, 28, 27, 26, 25, 24, 23, 22, 21, 20, 19, 18, 17, 16, 15, 14, 13, 12, 11, 10, 9, 8, 7, 6, 5, 4, 3, or 2 days. Combinations of any of these are also possible, e.g., tₘₐₓ may be between 1 and 3 days, or between 4 and 6 days, etc. Without wishing to be bound by any theory, it is believed that this may be due to the relatively high viscosity of the composition and/or slower release kinetics, since clindamycin typically in the blood of the subject has a plasma half-life of less than 24 hours.

In addition, in some cases, the active ingredient may be determined in the blood, and/or in the vagina of a subject after relatively long periods of time. For example, without wishing to be bound by any theory, it is believed that active ingredients such as clindamycin may have a relatively short half-life in the bloodstream, and compositions such as those described herein may release the active ingredient on relatively long time scales, thereby improving the availability of the active ingredient. Thus, for example, the active ingredient may be determined within the blood and/or within the vagina of a subject over a period of days after administration of the composition, e.g., after 1, 2, 3, 4, 5, 6, 7, 8, 9, 10, etc. days after the composition has been administered to the subject.

Without wishing to be bound by any theory, it is believed that in certain cases, the active ingredient may be present in the vagina and/or in the blood for times in excess of its half-life within the vagina and/or the blood, due to its continuing release from the composition, e.g., as applied to the vagina of the subject. For instance, the composition may be present within the vagina and/or in the blood for times of at least 2, at least 3, at least 5, at least 8, at least 10, at least 12, at least 15, at least 18, at least 20, at least 25, at least 30, at least 35, at least 40, at least 45, at least 50, etc. or more relative the composition's half-life within the vagina and/or within the blood.

As a non-limiting example, clindamycin has a half-life in the blood of less than 4 hours, but certain compositions such as described herein may release clindamycin, e.g., into the vagina of a subject, such that it can be detected within the vagina and/or within the blood 1 day after administration, 2 days after administration, 3 days after administration, 4 days after administration, 5 days after administration, 6 days after administration, etc.

The active ingredient may be present within the composition at any suitable concentration. For example, the active ingredient may be present at at least 0.1 wt %, at least 0.2 wt %, at least 0.3 wt %, at least 0.4 wt %, at least 0.5 wt %, at least 0.7 wt %, at least 1 wt %, at least 1.5 wt %, at least 2 wt %, at least 2.5 wt %, at least 3 wt %, at least 3.5 wt %, at least 4 wt %, at least 4.5 wt %, at least 5 wt %, at least 5.5 wt %, at least 6 wt %, at least 6.5 wt %, at least 7 wt %, at least 7.5 wt %, at least 8 wt %, at least 8.5 wt %, at least 9 wt %, at least 9.5 wt %, at least 10 wt %, etc. In addition, in some cases, the active ingredient may be present at no more than 10 wt %, no more than 9.5 wt %, no more than 9 wt %, no more than 8.5 wt %, no more than 8 wt %, no more than 7.5 wt %, no more than 7 wt %, no more than 6.5 wt %, no more than 6 wt %, no more than 5.5 wt %, no more than 5 wt %, no more than 4.5 wt %, no more than 4 wt %, no more than 3.5 wt %, no more than 3 wt %, no more than 2.5 wt %, no more than 2 wt %, no more than 1.5 wt %, no more than 1 wt %, no more than 0.8 wt %, no more than 0.6 wt %, no more than 0.4 wt %, no more than 0.2 wt %, etc. In addition, in certain instances, combinations of any of these ranges are also possible. For example, the active ingredient may be present at between 1 wt % and 5 wt %, between 0.5 wt % and 2 wt %, between 0.5 wt % and 5 wt %, or the like.

In addition, in accordance with certain embodiments, the composition may release the active ingredient over an extended period of time. In some cases, this may be determining by determining a concentration of an active ingredient in the mucus of the vagina after a certain period of time, for instance, after 1 day, 2 days, 3 days, etc. For example, in one set of embodiments, the concentration of the active ingredient may be at least 100 micrograms/g, at least 200 micrograms/g, at least 300 micrograms/g, at least 400 micrograms/g, at least 500 micrograms/g, at least 600 micrograms/g, at least 700 micrograms/g, etc. The mucus may be sampled, for example, using a swab, or other techniques known to those of ordinary skill in the art.

Other components may be present as well within a composition. As a non-limiting example, in one set of embodiments, the composition may include citrate and/or a citrate salt. These may include, for example, citric acid, citric acid monohydrate, sodium citrate, sodium citrate dihydrate, or the like. Other examples include other suitable salts, e.g., to make citrate buffer such as sodium phosphate, potassium phosphate, or the like. Buffers such as these may be used, for example, to maintain the pH of the composition (for example, at around 4.5, or another suitable pH). As another example, the composition may include benzyl alcohol. Benzyl alcohol may be useful, for example, as a solvent or a preservative.

Components such as these may each independently be present in any suitable amount or concentration. For example, a component may be present at at least 0.1 wt %, at least 0.2 wt %, at least 0.3 wt %, at least 0.4 wt %, at least 0.5 wt %, at least 0.7 wt %, at least 1 wt %, at least 1.5 wt %, at least 2 wt %, at least 2.5 wt %, at least 3 wt %, at least 3.5 wt %, at least 4 wt %, at least 4.5 wt %, at least 5 wt %, at least 5.5 wt %, at least 6 wt %, at least 6.5 wt %, at least 7 wt %, at least 7.5 wt %, at least 8 wt %, at least 8.5 wt %, at least 9 wt %, at least 9.5 wt %, at least 10 wt %, etc. In addition, in some cases, a component may be present at no more than 10 wt %, no more than 9.5 wt %, no more than 9 wt %, no more than 8.5 wt %, no more than 8 wt %, no more than 7.5 wt %, no more than 7 wt %, no more than 6.5 wt %, no more than 6 wt %, no more than 5.5 wt %, no more than 5 wt %, no more than 4.5 wt %, no more than 4 wt %, no more than 3.5 wt %, no more than 3 wt %, no more than 2.5 wt %, no more than 2 wt %, no more than 1.5 wt %, no more than 1 wt %, no more than 0.8 wt %, no more than 0.6 wt %, no more than 0.4 wt %, no more than 0.2 wt %, etc. In addition, in certain instances, combinations of any of these ranges are also possible. For example, the component may be present at between 1 wt % and 5 wt %, between 0.5 wt % and 2 wt %, between 0.5 wt % and 5 wt %, between 0.5 wt % and 2 wt %, or the like.

In addition, in one set of embodiments, water may be present within the composition. Any suitable amount of water may be present, for example, such that the composition forms a gel, has a relatively high viscosity as discussed herein, or the like. For example, in some cases, at least 50 wt %, at least 55 wt %, at least 60 wt %, at least 65 wt %, at least 70 wt %, at least 75 wt %, at least 80 wt %, at least 85 wt %, at least 90 wt %, or at least 95 wt % of the composition may be water.

In one set of embodiments, the composition is a gel. The can may be semi-solid material that includes a relatively large amount or concentration of water, e.g., as noted above. In some cases, the polymer (e.g., one or more poloxamers) may from a scaffold structure that contains the water within the gel.

The gel or other composition, e.g., as described herein, may have a relatively high viscosity, at least in one set of embodiments. Those of ordinary skill in the art will be aware of techniques for determining viscosity of a sample, for example, using devices such as a rheometers, viscometers, etc.

In some cases, the composition may have a viscosity at room temperature of at least 1 million cP, at least 1.1 million cP, at least 1.2 million cP, at least 1.3 million cP, at least 1.4 million cP, at least 1.5 million, at least 1.6 million cP, at least 1.8 million cP, at least 2 million cP, at least 2.2 million cP, at least 2.4 million cP, at least 2.6 million cP, at least 2.8 million cP, at least 3 million cP, at least 3.5 million cP, at least 4 million cP, etc. In addition, in certain embodiments, the composition may have a viscosity of no more than 4 million cP, no more than 3.5 million cP, no more than 3 million cP, no more than 2.8 million cP, no more than 2.6 million cP, no more than 2.4 million cP, no more than 2.2 million cP, no more than 2.0 million cP, no more than 1.8 million cP, no more than 1.6 million cP, no more than 1.5 million cP, no more than 1.4 million cP, no more than 1.3 million cP, no more than 1.2 million cP, no more than 1.1 million cP, no more than 1.0 million cP, etc. Combinations of any of these are also possible, for example, the composition may exhibit a viscosity of between 1.5 million cP and 2 million cP, between 1.8 million cP and 2.4 million cP, between 1.2 million cP and 3 million cP, etc.

In some cases, the composition may contain xanthan gum and/or another stabilization polymer, and a polymer such as a poloxamer, which may cause the composition to have a relatively high viscosity. In some embodiments, the composition may contain no other component that changes the viscosity of said composition at room temperature by more than +/−100,000 centipoise.

In addition, in one set of embodiments, the composition may have a relatively low concentration or amount of air. For instance, in one embodiment, the composition is substantially free of air. In some cases, during manufacture, a large amount of air may be introduced into the composition, e.g., as foam or bubbles, etc. However, this air may be undesirable in accordance with certain embodiments, and accordingly, the composition may be prepared by also including a step of removing air from the composition that has been introduced during manufacture.

In some embodiments, the composition, after removing at least some of the air may contain no more than 20 vol %, no more than 15 vol %, no more than 12 vol %, no more than 10 vol %, no more than 8 vol %, no more than 6 vol %, no more than 5 vol %, no more than 4 vol %, no more than 3 vol %, no more than 2 vol %, or no more than 1 vol %, etc. of air. In some cases, the air may be removed such that no air bubbles are visually present within the composition.

Without wishing to be bound by any theory, it is believed that the presence of air may reduce the viscosity of the composition, e.g., making it easier for the composition to flow. Accordingly, in some embodiments, any air that is introduced may be removed, thereby increasing the viscosity of the composition, e.g., to at least 1 million cP, or other ranges of viscosities, such as any of those described herein. Furthermore, in some cases, removing the air may also increase the density of the final composition. For instance, the density of the composition may be at least 0.98 g/cm³, at least 0.99 g/cm³, at least 1 g/cm³, at least 1.01 g/cm³, at least 1.02 g/cm³, at least 1.03 g/cm³, at least 1.05 g/cm³, at least 1.1 g/cm³, etc. In some cases, the density of the composition may be no more than 1.1 g/cm³, no more than 1.05 g/cm³, no more than 1.03 g/cm³, no more than 1.02 g/cm³, no more than 1.01 g/cm³, etc. Furthermore, in some embodiments, combinations of any of these ranges are possible. For example, the final density of the composition may be between 1.00 g/cm³ and 1.01 g/cm³, between 0.99 g/cm³ and 1.02 g/cm³, etc.

In one embodiment, air may be removed from a composition, e.g., during or after formation, by applying a pressure less than atmospheric or ambient pressure to the composition. For instance, the pressure that is applied may be less than 1 bar, less than 800 mbar, less than 600 mbar, less than 500 mbar, less than 400 mbar, less than 300 mbar, less than 200 mbar, less than 100 mbar, less than 75 mbar, less than 60 mbar, less than 50 mbar, less than 40 mbar, less than 30 mbar, less than 20 mbar, less than 10 mbar, less than 5 mbar, less than 3 mbar, less than 2 mbar, less than 1 mbar, etc. It should be noted that 1 atmosphere is approximately 1 bar, and that these pressures are absolute pressures (i.e., a pressure of less than about 1 bar means a pressure lower than atmospheric pressure, i.e., a vacuum pressure). Such pressures may be applied for any suitable length of time, e.g., at least 10 min, at least 20 min, at least 30 min, at least 45 min, at least 1 h, at least 2 h, at least 3 h, at least 4 h, at least 6 h, at least 24 h, at least 1 day, etc. In addition, in some cases, the pressure may be applied until the composition comprises less than a certain amount of air, e.g., less than 15 vol %, or other percentages such as those described herein. As yet another example, in some cases, a solution may be caused to form a gel by removing a certain amount of air from the solution.

In certain embodiments, as another example, the air may be removed from the composition using a Versator. In a Versator, a material such as a liquid is spread onto the inside of a rotating Versator disc under vacuum to remove entrapped air, foam, gas, etc. While the liquid travels across the disc, the high vacuum draws off the bubbles, etc., from the liquid. Versators may be obtained from several commercial sources. Accordingly, in some embodiments, a composition such as described herein may be treated using a Versator for any suitable length of time, e.g., at least 10 min, at least 20 min, at least 30 min, at least 45 min, at least 1 h, at least 2 h, at least 3 h, at least 4 h, at least 6 h, at least 24 h, at least 1 day, etc., and/or until the composition comprises less than a certain amount of air, e.g., less than 15 vol %, or other percentages such as those described herein

As yet another example, in some cases, the air may be removed from the composition using centrifugation. Without wishing to be bound by any theory, it is believed that by centrifuging the composition, air (being less dense) may be forced out of the composition. Thus, for example, the material may be centrifuged at any suitable speed, e.g., at least at least 500 RPM, at least 1,000 RPM, at least 2,000 RPM, at least 3,000 RPM, at least 5,000, at least 10,000 RPM, or the like, for any suitable length of time, e.g., at least 10 min, at least 20 min, at least 30 min, at least 45 min, at least 1 h, at least 2 h, at least 3 h, at least 4 h, at least 6 h, at least 24 h, at least 1 day, etc., and/or until the composition comprises less than a certain amount of air, e.g., less than 15 vol %, or other percentages such as those described herein.

Furthermore, it should be understood that these techniques are non-limiting, and that other methods of removing air, besides centrifuges or Versators, are also possible in still other embodiments.

Accordingly, as discussed above, a variety of compositions are covered in various embodiments, including any suitable combinations of any of the above-described components, such as poloxamer, xanthan gum and/or another stabilization polymer, an active ingredient for treating bacterial vaginosis, and water. For example, in one set of embodiments, at least 50 wt %, at least 60 wt %, at least 70 wt %, at least 80 wt %, at least 90 wt %, at least 95 wt %, or at least 99 wt % of the composition comprises or consists essentially of polymer such as a poloxamer, xanthan gum and/or another stabilization polymer, an active ingredient for treating bacterial vaginosis, and water.

In another set of embodiments, the composition comprises poloxamer 407 at 10-20 wt %, xanthan gum at 1-5 wt %, clindamycin and/or a salt thereof at 1-5 wt %, citrate and/or a citrate salt at 0.5-2 wt %, benzyl alcohol at 0.5-5 wt %, and water at 60-90 wt %. The composition, in still another set of embodiments, comprises a poloxamer at 10-20 wt %, xanthan gum at 1-5 wt %; and an active ingredient for treating bacterial vaginosis at 1-5 wt %. In yet another set of embodiments, the composition consists essentially of the poloxamer 407, the xanthan gum, the clindamycin and/or salt thereof, the citrate and/or a citrate salt, the benzyl alcohol, and water.

In addition, in one set of embodiments, the composition is a gel comprising poloxamer 407 at 10-20 wt %, xanthan gum at 1-5 wt %, clindamycin and/or a salt thereof at 1-5 wt %, citrate and/or a citrate salt at 0.5-2 wt %, and benzyl alcohol at 0.5-5 wt %. In another set of embodiments, the gel comprises poloxamer 407 at 10-20 wt %, xanthan gum at 1-5 wt %, clindamycin and/or a salt thereof at 1-5 wt %, citrate and/or a citrate salt at 0.5-2 wt %, and benzyl alcohol at 0.5-5 wt %.

Furthermore, as mentioned, certain aspects as described herein are generally directed to compositions and methods for applying such compositions for the treatment or prevention of bacterial vaginosis or other indications, e.g., to the vagina of a subject, such as a human. The subject can also be a non-human animal.

In certain cases, the bacterial vaginosis may be recurrent bacterial vaginosis. Recurrent bacterial vaginosis may be bacterial vaginosis that occurs repeatedly, e.g., as determined by at least 3 separate diagnoses of bacterial vaginosis within a 365-day period within a subject. In some cases, subjects having bacterial vaginosis may have bacteria that is difficult to treat with other methods. However, as discussed herein, certain embodiments as discussed herein may be directed to compositions and methods for applying such compositions to the vagina of a subject to treat recurrent bacterial vaginosis.

In addition to bacterial vaginosis, in other embodiments, indications that can be treated with a composition such as those described herein include, but are not limited to, vulvovaginal candidiasis (yeast infection), vulvovaginal pain, cervical or vaginal cancer, hormone therapy, etc.

In one set of embodiments, a composition such as discussed herein may be used for the treatment of subjects having or at risk of bacterial vaginosis, or other indications, e.g., by being applied to the vagina of a subject. As discussed herein, it is believed that such compositions, when applied to the vagina of a subject, may not readily exit the vagina, e.g., due to their viscosities. This may facilitate better outcomes, as the composition is able to treat the bacteria, e.g., due to the release or proximity of active ingredients within the composition for treating bacterial vaginosis, for example, clindamycin and/or a salt thereof, or other compound such as described herein. In some embodiments, such treatments may result in improved subject outcomes, e.g., as discussed herein. In addition, in certain cases, improvements in vaginal health may be observed relatively quickly, for example, within 3 days, within 2 days, or within 1 day of treatment.

In addition, in some embodiments, the subject may not necessarily be a subject that has bacterial vaginosis, or another indication, but instead may be a subject that is at risk for bacterial vaginosis. In some cases, for instance, a composition such as is described herein may be administered to the vagina of a subject prophylactically. For instance, subjects who may be about to engage in sex, douching, bathing (e.g., bubble baths), using a vaginal deodorant, or other activities that may result in an increased risk of bacterial vaginosis, or another indication, may have the composition applied in association with such activities, e.g., before, during, or after such activities, e.g., to prevent or reduce potential bacterial vaginosis (or other indications, such as those described herein) in the subject.

Terms such as “treat,” “treatment,” “treating,” etc. comprise therapeutic treatment of subjects having already developed a disease, in particular in manifest form. Therapeutic treatment may be symptomatic treatment in order to relieve the signs and/or symptoms of the disease or causal treatment in order to reverse, partially reverse, stop, or slow down the progression of the disease. Thus, the compositions and methods of the present disclosure may be used, for instance, as therapeutic treatment (e.g., for acute or chronic therapy).

Additionally, terms such as “prevent,” “preventing,” or “prevention” generally refer to the reduction of the occurrence of the disease, and/or a sign and/or symptom thereof, in the treated sample relative to an untreated control sample, or delays the onset of one or more signs and/or symptoms of the disease relative to the untreated control sample, in a statistically significant manner. Preventing the disease, and/or a sign and/or a symptom thereof, includes preventing or delaying the initiation of the disease, sign, and/or symptom. Prevention also includes preventing a recurrence of the disease, sign, and/or symptom.

In one set of embodiments, for example, certain composition such as those described herein may be used to reduce vaginal discharge in a subject. As an example, subjects having bacterial vaginosis, or other indications may experience relatively large amounts of vaginal discharge. While healthy vaginas exhibit some amounts of discharge, those having bacterial vaginosis may exhibit significantly greater amounts of vaginal discharge, and/or changes in the appearance or nature of the vaginal discharge. For instance, the discharge may exhibit changes or differences in color, consistency, odor, etc., as compared to normal or healthy vaginal discharge. For example, the discharge may be more white, yellow, green, or gray than normal.

Treatments such as discussed herein may return the vaginal discharge to normal or healthy amounts and/or appearance in a subject. For instance, there may be a reduction in volume of the vaginal discharge of at least 25 vol %, at least 30 vol %, at least 40 vol %, at least 50 vol %, at least 60 vol %, at least 70 vol %, at least 80 vol %, or at least 90 vol % after treatment begins. The volume of vaginal discharge may be determined qualitatively (e.g., by the subject) or quantitatively (e.g., by collecting the amount of discharge and measuring its volume or mass, by collecting samples from the vagina at regular time intervals, etc.). In addition, in some embodiments, after treatment as discussed herein, the subject may no longer exhibit relatively large amounts of vaginal discharge, e.g., any subsequent vaginal discharge that is observed would be considered to be normal or healthy. In addition, in some embodiments, the decrease in discharge may be relatively rapid. For instance, there may be a significantly less amount of vaginal discharge that can be determined by 1 day after treatment, by 2 days after treatment, by 3 days after treatment, etc.

In another set of embodiments, certain compositions such as those described herein may be used to reduce vaginal odor. Bacterial vaginosis may result in vaginal discharge that has a relatively strong, unpleasant odor, which is sometimes described as a “fishy” smell. Without wishing to be bound by any theory, it is believed that such odors may be caused in some cases by various chemicals produced during bacterial infection. For example, such bacteria may produce amines such as trimethylamine, which may be perceived as a “fishy” smell. Such amines or other chemicals may exit the vagina, e.g., through vaginal discharge, which can be perceived as such a smell. In some cases, even relatively low concentrations of such chemicals may be detectable as an increased odor, e.g., relative to normal or healthy vaginal discharge, which typically has a relatively mild odor. For instance, the concentration of trimethylamine may be detectable during bacterial vaginosis at concentrations of at least 0.1 ppb, at least 0.3 ppb, at least 0.5 ppb, at least 1 ppb, at least 3 ppb, at least 5 ppb, at least 10 ppb, at least 30 ppb, at least 50 ppb, at least 100 ppb, at least 300 ppb, at least 500 ppb, at least 1000 ppb, etc., (by volume).

Accordingly, in certain embodiments, treatments such as those discussed herein may result in the decreased production of odors, e.g., those caused by amines such as trimethylamine, for instance, due to treatment of bacteria within the vagina. In some cases, the concentration of such chemicals within the vaginal discharge may decrease by at least 5-fold, at least 10-fold, at least 50-fold, at least 100-fold, at least 500-fold, at least 1000-fold, etc., as compared to before treatment. This may thus result in a significant decrease in the intensity of the odor. Such decreases in odor may be determined, for example, qualitatively (e.g., by the subject) or quantitatively (e.g., by determining a concentration of amines, such as trimethylamine, within the vaginal discharge). In addition, in some embodiments, the decrease in odor may be relatively rapid. For instance, there may a determinable decrease in the odor intensity, qualitatively and/or quantitatively, by 1 day after treatment, by 2 days after treatment, by 3 days after treatment, etc.

In certain embodiments, certain compositions such as those described herein may be used to improve the pH of the vagina. This may be important as a measure of vaginal health. For example, the pH of a healthy vagina is typically between about 3.8 and about 4.5. However, in bacterial vaginosis, the pH may be relatively elevated (i.e., less acidic). Thus, according to certain embodiments, treatments such as those discussed herein may result in a decrease of the pH of the vagina (i.e., more acidic], e.g., to pH values of less than 6, less than 5.5, less than 5, or less than 4.5. Furthermore, according to certain embodiments, the decrease in pH may be relatively rapid. For instance, there may a determinable decrease in pH, e.g., to pH values such as these, by 1 day after treatment, by 2 days after treatment, by 3 days after treatment, etc.

In some embodiments, treatment using certain compositions such as those described herein may result in a decrease in clue cells. As would be understood by those of ordinary skill in the art, clue cells are generally identified as epithelial cells of the vagina that may exhibit a distinctive stippled appearance when being covered or infected with bacteria. Such clue cells may be an indication of the bacterial load within the vagina, and they can be used to predict vaginal health. In some cases, vaginal samples (e.g., vaginal discharge or vaginal swabs, etc.] may be collected and analyzed for the presence of clue cells. For example, a swab of vaginal material may be mounted on a glass slide and examined under a light microscope. Clue cells may appear as vaginal epithelial cells with a coating of bacteria that may obscure their peripheral borders when viewed at relatively high magnifications (e.g., 400×). Those of ordinary skill in the art will be aware of techniques for observing and counting clue cells.

Accordingly, in certain embodiments, treatments such as those discussed herein may result in decreased populations of clue cells in the vagina. In some cases, there may be a decrease of at least 50%, at least 60%, at least 70%, at least 80%, at least 90%, or at least 95%, etc., in the number of clue cells present within the vagina, e.g., as determined from sampling using vaginal swabs or other techniques. In addition, in some embodiments, the decrease in clue cells may be relatively rapid. For instance, there may a determinable decrease in the odor intensity, qualitatively and/or quantitatively, by 1 day after treatment, by 2 days after treatment, by 3 days after treatment, etc.

In some cases, systems and methods such as those described herein may lead to improved vaginal health, and in some embodiments, relatively quickly, for example, within 3 days, within 2 days, or within 1 day of treatment. In some cases, improved vaginal health may be determined using tests such as the Nugent test or Amsel's criteria. For example, improvements may be determined in pH, clue cell numbers, vaginal discharge amounts and/or quality, and/or decreases in vaginal odor. In some cases, for example, improved vaginal function may be determined by determining a decrease in a Nugent score, which is a Gram stain scoring system used to determine bacterial vaginosis. High Nugent scores, i.e., above 7, may indicate bacterial vaginosis in a subject. However, after treatment such as described herein, the Nugent score may decrease to below 7. In addition, in some embodiments, such changes in Nugent score may be relatively rapid, e.g., by 1 day after treatment, by 2 days after treatment, by 3 days after treatment, etc.

As another example, in some cases, treatment using certain compositions such as those described herein may result in sufficient improvement of a subject such that the subject no longer meets Amsel's criteria for bacterial vaginosis, for example, within such time periods. Amsel's criteria includes the determination of various parameters such as vaginal pH, the presence of clue cells, abnormal vaginal discharge, and the release of an amine or “fishy” odor after the addition of 10% potassium hydroxide to a sample, e.g., of vaginal fluid or mucus.

In certain aspects, the composition can be applied to a subject, e.g., to the vagina of a subject, and/or to another body cavity, for example, the mouth or the rectum. Any suitable technique may be used to apply the composition to the subject. For instance, the composition may be free or mass flowing, e.g., so that it may be administered through an applicator or other suitable device. Thus, in some embodiments, the composition may be contained within applicator, such as a vaginal applicator or a syringe, which can be applied, e.g., by the subject, or by another person.

The subject may be, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., monkeys such as cynomolgus monkeys or rhesus monkeys, chimpanzees, etc.); commercially relevant mammals such as cattle, pigs, horses, sheep, rabbits, mice, rats, goats, cats, dogs, etc.) and birds (e.g., commercially relevant birds such as chickens, ducks, geese, turkeys, etc.). In certain embodiments, the subject is a mammal. The subject may be a male or female and at any stage of development. A non-human animal may be a transgenic animal.

In one set of embodiments, as discussed, the composition is applied to treat the subject with a therapeutically effective amount of an active ingredient, such as any of those described herein. The therapeutically effective amount may be an amount which, when administered to a subject for treating or preventing a disease, is sufficient to effect such treatment or prevention for the disease, for example, bacterial vaginosis or another disease. Examples include, but are not limited to, vulvovaginal candidiasis (yeast infection), vulvovaginal pain, cervical or vaginal cancer, hormone therapy, or the like, e.g., as discussed herein. For example, the subject may have or be at risk for bacterial vaginosis or another disease. The therapeutically effective amount may also be an amount sufficient to elicit a desired biological response, i.e., alleviating a symptom. The therapeutically effective amount may vary depending on such factors as the desired biological endpoint, the mode of administration, and/or the age and health of the subject.

Still another aspect of the present disclosure is directed to kits. The kit may include a package or an assembly including one or more of the compositions as described herein, and/or other compositions. Each of the compositions of the kit may be provided in liquid form (e.g., in solution), or in solid form (e.g., a dried powder), or in gaseous form in some cases. In certain cases, some of the compositions may be constitutable or otherwise processable (e.g., to an active form), for example, by the addition of a suitable solvent or other species, which may or may not be provided with the kit. Examples of other components include, but are not limited to, solvents, surfactants, diluents, salts, buffers, emulsifiers, chelating agents, fillers, antioxidants, binding agents, bulking agents, preservatives, drying agents, antimicrobials, needles, syringes, packaging materials, tubes, bottles, flasks, beakers, dishes, frits, filters, rings, clamps, wraps, patches, containers, and the like, for example, for using, administering, modifying, assembling, storing, packaging, preparing, mixing, diluting, and/or preserving the compositions components for a particular use, for example, to a sample and/or a subject.

A kit may, in some cases, include instructions in any form that are provided in connection with the compositions described herein in such a manner that one of ordinary skill in the art would recognize that the instructions are to be associated with those compositions. For instance, the instructions may include instructions for the use, modification, mixing, diluting, preserving, administering, assembly, storage, packaging, and/or preparation of the compositions and/or other compositions associated with the kit. In some cases, the instructions may also include instructions for the delivery and/or administration of the compositions, for example, for a particular use, e.g., to a sample and/or a subject. The instructions may be provided in any form recognizable by one of ordinary skill in the art as a suitable vehicle for containing such instructions, for example, written or published, verbal, audible (e.g., telephonic), digital, optical, visual (e.g., videotape, DVD, etc.) or electronic communications (including Internet or web-based communications), provided in any manner.

The following examples are intended to illustrate certain embodiments of the present disclosure, but do not exemplify the full scope of the disclosure.

### Example 1

A composition in accordance with one embodiment is as follows (all components USP grade):

TABLE 3

| Material | g/100 g |
|---|---|
| Poloxamer 407 | 16.50 |
| Xanthan gum | 2.00 |
| Clindamycin phosphate | 2.00 |
| Citric acid monohydrate | 0.60 |
| Sodium citrate dihydrate | 0.55 |
| Benzyl alcohol | 1.00 |
| Purified water (qs) | 77.35 |
| Total | 100 |

### Example 2

This example illustrates a method of making the composition described in Example 1, in accordance with another embodiment.

Purified water is added to a kettle (or other suitable container), which is placed under a dissolver (30-60 HP) with a 12-inch stand dissolver blade. The dissolver is started, and clindamycin phosphate is added to the kettle. The dissolver is allowed to mix for at least 10 minutes, or until the clindamycin phosphate has visually dissolved in the water.

Afterwards, mixing is continued while citric acid monohydrate and sodium citrate dihydrate are added to the kettle. The kettle is then cooled with chilled cooling water, e.g., a temperature of 8° C. Mixing is continued for at least 5 minutes, or until these have visually dissolved.

Mixing and cooling then continues while benzyl alcohol and poloxamer 407 is added. These are mixed until the water is clear and everything has visually dissolved.

Mixing and cooling continues while xanthan gum is slowly added. Mixing of the xanthan gum continues for at least 10 minutes, or until the composition is visually uniform. The speed of mixing may be adjusted, for example, as the composition thickens and its viscosity increases, and/or to avoid trapping too many air bubbles within the composition.

Next, the composition is transferred into a round-bottom, jacketed, stainless steel pressure/vacuum kettle, or another suitable container. The kettle is cooled as before, e.g., using chilled cooling water at 8° C. Mixing in the kettle is started and the batch is slowly recirculated under a vacuum. Mixing, cooling with chilled cooling water, and recirculating under vacuum occurs for at least 30 minutes, or until the composition has been sufficiently deaerated.

It should be understood that the above is by way of example only, and that other techniques for making the compositions described herein are also contemplated. For example, the addition sequence of poloxamer 407 and xanthan gum may be reversed, with the poloxamer going into the mixing first. Air may be removed using a centrifuge, a Versator, or the like.

### Example 3

This example illustrates a method of making a composition in accordance with another embodiment.

The primary compounding phase in this example is prepared by adding 154 kg of Purified Water USP to a 270 L kettle. The kettle is placed under a dissolver equipped with a 10-inch standard blade. Mixing commences at about 600 rpm. 4.86 kg of clindamycin phosphate is added and mixed for 10 minutes until visually dissolved. Mixing continues and 1200 g citric acid and 1100 g of sodium citrate are added and mixed for 6 min until visually dissolved. Cooling of the batch is started during this step by flowing chilled water through the kettle jacket.

Mixing continues and 2000 g of benzyl alcohol and 33.0 kg of poloxamer 407 are added. Cooling and mixing continue until the solution is clear and the poloxamer is visually dissolved. The batch temperature reaches approximately 11° C. during this step. This step creates foam on the surface of the batch and the mixing speed is decreased to allow the foam to dissipate. Mixing speed is then increased, eventually reaching 1200 rpm for addition of the 4.00 kg of xanthan gum. After the addition of the xanthan gum, the product rapidly increases in viscosity and the original mixing blade size is switched to a 12-inch blade to better facilitate mixing at this stage of the process. A 14-inch blade may also be used at this scale.

After this step in the process, the product has generated a large amount of foaming. The foaming can be reduced by transferring the product into a cooling, jacketed vacuum vessel and recirculate the product for several passes to help remove the air. Another approach is to use a Versator, which is a device for removing air from liquids and semi-solids. The product was passed through the Versator several times until all air was removed. Measurements of the viscosity after Versator treatment rose to over 3 million cP, from 1.2 million cP at room temperature prior to using the Versator.

### Example 4

This is an example showing the viscosity of a composition in accordance with one embodiment as a function of temperature. It will be noted that, even at room temperature (about 25° C.), the composition has a viscosity of over 1 million cP.

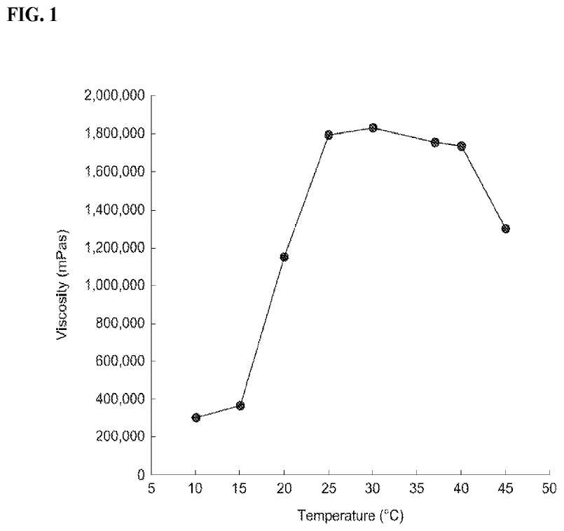

FIG. 1 illustrates the viscosity (in mPA s, equilavent to cP) plotted as a function of temperature for the composition shown in Example 1. Viscosity was measured using a Brookfield Viscometer.

In this experiment, the viscosity at 25° C. was found to be 1,798,000 cP, while the viscosity at 37° C. (body temperature) was found to be 1,759,000 cP.

### Example 5

A study of 290 subjects was evaluated in this example, and tested with either a placebo or a composition comprising clindamycin as discussed herein. The treatment group included 204 randomized subjects and 190 (93.1%) subjects who completed the study. The placebo treatment group included 103 randomized subjects and 100 (97.1%) subjects who completed the study. The majority of subjects who failed to complete the study were lost to follow-up, although the lost-to-follow-up rate was low (3.4% treatment subjects and 1.9% placebo subjects). The blind was not broken for any subjects during this study.

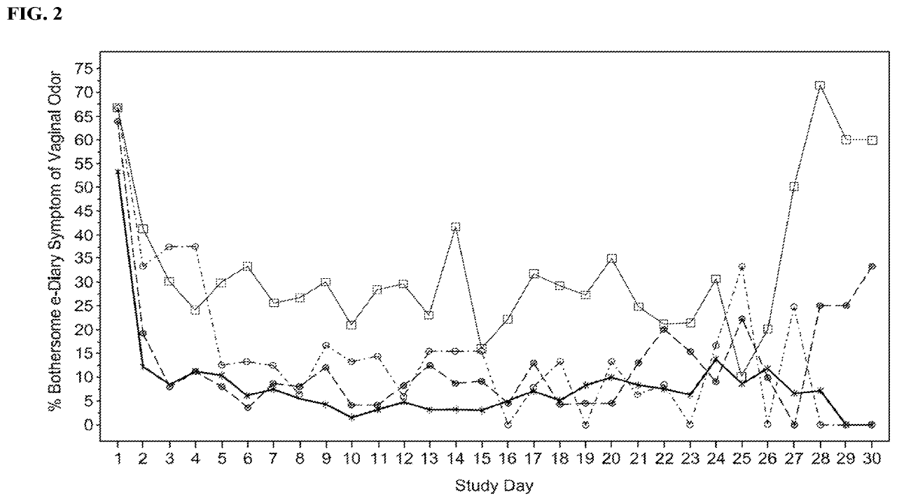

FIG. 2 is a graph showing the percent of subjects reporting either odor or discharge that they considered bothersome (mild, moderate, or severe), starting with the day of treatment. Those subjects who received placebo and did not experience a clinical cure (open boxes) were most likely to report continued bothersome symptoms. Those who received placebo and experienced a clinical cure (open circles) were still somewhat more likely to report bothersome odor than those who received the composition (whether clinically cured or not-stars and closed circles, respectively). Those who received the composition tended to report improvement in both odor and discharge, commencing shortly post dosing and continuing through the assessment period. In FIG. 2, it can be seen that subjects receiving treatment generally scored lower (better) than subjects receiving a placebo.

### Example 6

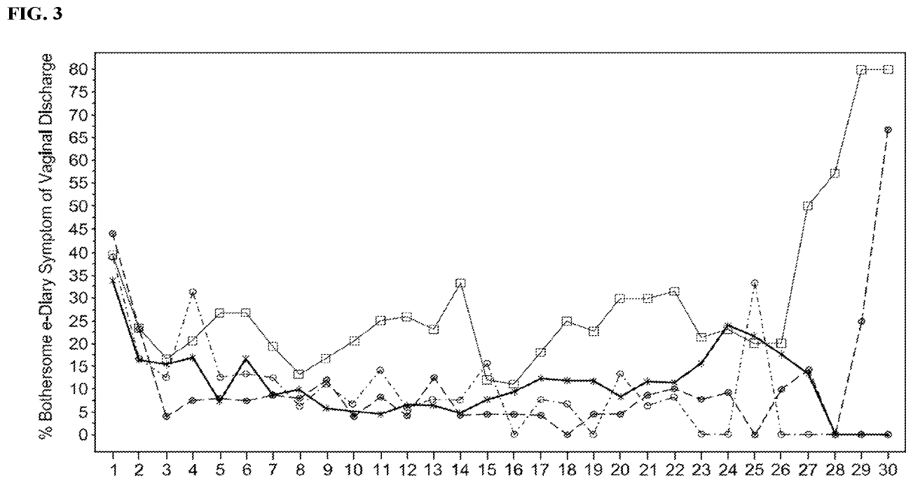

The subjects described in Example 5 were also tested for vaginal discharge. Results are shown in FIG. 3, which shows the percent of subjects reporting either odor or discharge that they considered bothersome (mild, moderate, or severe), starting with the day of treatment. Those subjects who received placebo and did not experience a clinical cure (open boxes) were most likely to report continued bothersome symptoms. Those who received placebo and experienced a clinical cure (open circles) were still somewhat more likely to report bothersome odor than those who received treatment (whether clinically cured or not—stars and closed circles). Those who

received treatment tended to report improvement in both odor and discharge, commencing shortly post dosing and continuing through the assessment period. Thus, in FIG. 3, it can be seen that subjects receiving treatment generally scored lower (better) than subjects receiving a placebo.

### Example 7

This example illustrates treatment of subjects with recurrent bacterial vaginosis, generally diagnosed as more than 3 episodes of bacterial vaginosis within the past 12 months. Subjects from Example 5 with more than 3 episodes of bacterial vaginosis in the prior 12 months were studied in this example. 57 subjects were identified. The clinical cure rate among placebo patients with a history of more than 3 prior episodes was found to be 25.0% (5 out of 20), while in comparison, the clinical cure rate among treatment subjects was found to be 75.7% (28 out of 37).

### Example 8

In this example, 21 healthy female subjects had PK (pharmacokinetic) samples collected daily through day 7 after treatment on day 1 with one full applicator (5 g) of clindamycin phosphate vaginal gel, in accordance with one embodiment.

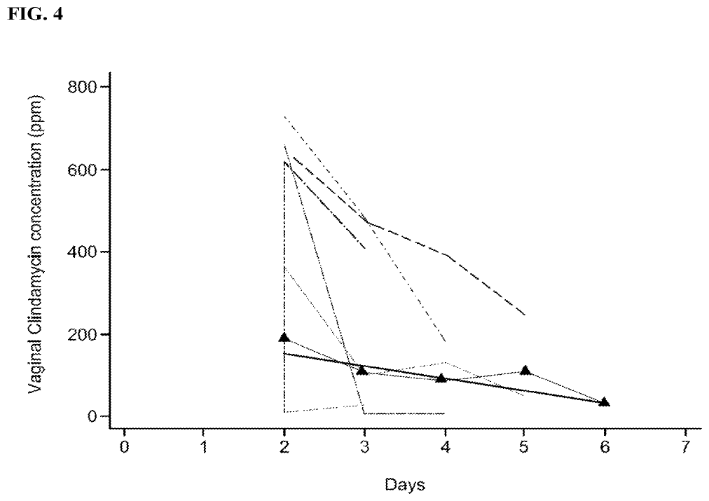

FIG. 4 illustrates the concentration of clindamycin in the vagina after treatment (in days) for several subjects. The average (geometric mean) is indicated by solid triangles. It was found that clindamycin was detectable from day 2 through day 7, averaged across the population in the study.

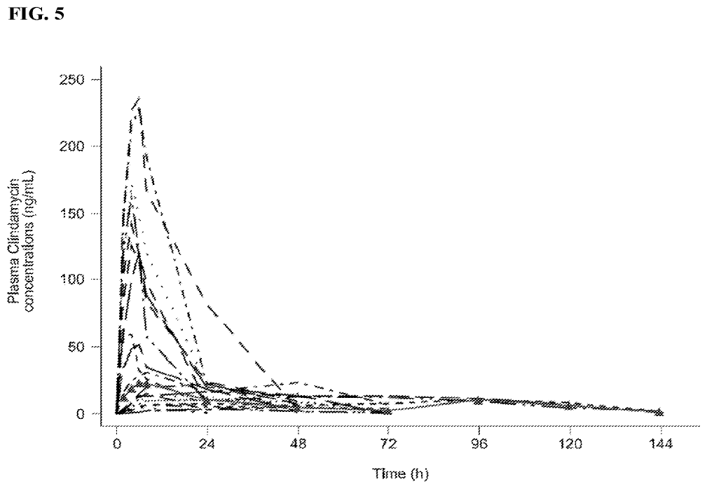

Similarly, FIG. 5 illustrates the concentration of clindamycin in the plasma after treatment (in days) for several subjects. The average (geometric mean) is indicated by solid triangles. It was found that clindamycin was detectable up to 144 hours in the plasma, averaged across the population in the study.

### Example 9

A study was conducted where a formulation of one embodiment of the present disclosure was provided to 109 subjects, while a placebo was presented to 50 subjects. The subjects were told to administer the formulation, and were asked 3 questions, as follows:
6a. Compared to other vaginally applied BV drugs, how was leakage experience with study treatment (much less bothersome/less bothersome/about the same/more bothersome/much more bothersome)?
6b. Compared to all other vaginally applied products you have used for BV, how was overall experience with study treatment (much better/better/neither better nor worse/worse/much worse)?
6c. Compared to all other vaginally applied products you have used for BV, how was cleanliness experience with safety treatment (much cleaner/slightly cleaner/neither cleaner nor messier/slightly messier/much messier)?

The formulation included clindamycin phosphate vaginal gel, 2%, and was supplied in a 25 g tube with an accompanying applicator and instructions for use. To dispense the product, the subject screwed the applicator onto the tube and expressed product from the tube into the applicator, until the stop line on the plunger was visible just beyond the applicator barrel. One applicator (5 g) of clindamycin phosphate vaginal gel, 2% (100 mg clindamycin), filled to the target fill line on the plunger, would contain approximately 5.2 g of study product, in order to dispense the target 5 g dose vaginally within 1 day of randomization.

Placebo vaginal gel (HEC Universal Placebo Gel) was supplied in a matching 25 g tube with an accompanying applicator, as above. One applicator of placebo gel, filled to the target fill line on the plunger, would contain approximately 5.2 g of study product, in order to dispense the target 5 g dose vaginally within 1 day of randomization.

The results of these questions are shown in Table 4. It can be concluded that there was significantly less leakage of the tested embodiment, as compared to other vaginally applied BV drugs that the subjects being tested had used previously.

TABLE 4

| | DARE-BV1 | | Placebo | | Total | | |
|---|---|---|---|---|---|---|---|
| Question | (N = 203) | | (N = 103) | | (N = 306) | | |
| Answer | n (%) | | n (%) | | n (%) | | P-Value |
| 6a. Compared to other vaginally | | | | | | | 0.912 |
| applied BV drugs, how was leakage | | | | | | | |
| experience with study treatment | | | | | | | |
| n | 109 | | 50 | | 159 | | |
| Much less bothersome | 39 | (35.8) | 17 | (34.0) | 56 | (35.2) | |
| Less bothersome | 22 | (20.2) | 9 | (18.0) | 31 | (19.5) | |
| About the same | 36 | (33.0) | 21 | (42.8) | 57 | (35.8) | |
| More bothersome | 9 | (8.3) | 3 | (6.0) | 12 | (7.5) | |
| Much more bothersome | 3 | (2.8) | 0 | | 3 | (1.9) | |
| 6b. Compared to all other vaginally | | | | | | | <0.001* |
| applied products you have used for | | | | | | | |
| BV, how was overall experience | | | | | | | |
| with study treatment | | | | | | | |
| n | 109 | | 50 | | 159 | | |
| Much better | 46 | (42.2) | 11 | (22.0) | 57 | (35.8) | |
| Better | 36 | (33.0) | 11 | (22.0) | 47 | (29.6) | |
| Neither better nor worse | 21 | (19.3) | 23 | (46.0) | 44 | (27.7) | |
| Worse | 5 | (4.5) | 4 | (6.0) | 9 | (5.7) | |
| Much worse | 1 | (0.9) | 1 | (2.0) | 2 | (1.3) | |

| 6c. Compared to all other vaginaly | | | | 0.145 |
|---|---|---|---|---|
| applied products you have used for | | | | |
| BV, how was cleanliness experience | | | | |
| with study treatment | | | | |
| n | 109 | 50 | 159 | |
| Much cleaner | 46 (42.2) | 15 (30.0) | 61 (38.4) | |
| Slightly cleaner | 22 (20.2) | 10 (20.0) | 32 (20.1) | |
| Neither cleaner nor messier | 36 (33.0) | 23 (46.0) | 59 (37.1) | |
| Slightly messier | 5 (4.6) | 2 (4.0) | 7 (4.4) | |
| Much messier | 0 | 0 | 0 | |

### Example 10

In this example, the PK (pharmacokinetics) profile of an embodiment was determined. In this example, 21 female subjects, aged 28 to 80 years old at the time of screening, were enrolled into this single-dose PK study and 20 subjects (95.2%) completed the study.

Clindamycin plasma concentrations were measured using a liquid chromatography/tandem mass spectrometry (LC-MS/MS) assay method that was qualified for limit of quantitation, method accuracy and precision, specificity, selectivity, solution and matrix stability, and robustness.

Clindamycin vaginal concentrations were measured using a high pressure liquid chromatography (HPLC) assay method that was qualified for linearity, limit of detection, limit of quantitation, system precision, accuracy, method precision, intermediate precision, specificity, solution stability, and robustness.

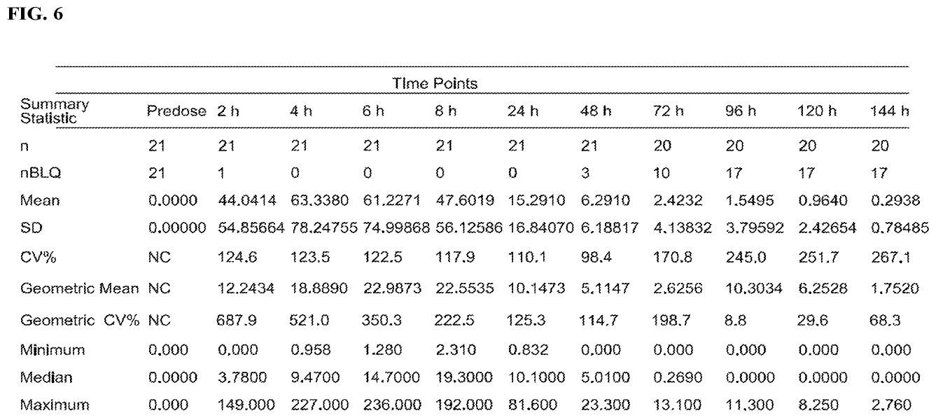

Blood sampling times and plasma concentration data for clindamycin are presented in FIG. 6, including mean (+/−SD) plasma clindamycin concentrations over time. Plasma clindamycin concentrations were quantifiable in all but 1 subject by the first post-dose sample at 2 hours, in all subjects by 4 hours post-dose, and were quantifiable in all subjects through 24 hours post-dose. At 48, 72, and 96 through 144 hours post-dose, 18, 10, and 3 subjects, respectively, had measurable concentrations of plasma clindamycin.

In FIG. 6, BLQ is Below the Lower Limit of Quantification; CV % is Coefficient of Variation; Geometric CV % is Geometric Coefficient of Variation; NC is Not Calculable; and SD is Standard Deviation. Concentration BLQ [<0.50 (ng/mL)] values were set to zero for calculation of summary statistics. n=number of subjects in the respective category.

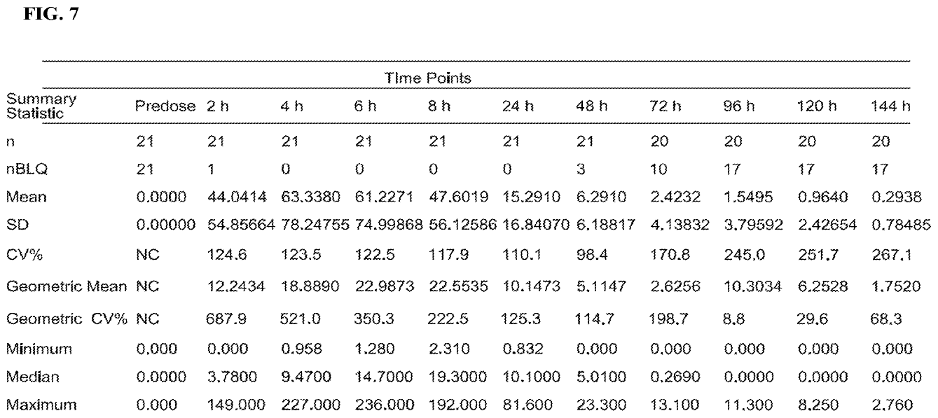

In addition, individual vaginal sampling times, weights, amounts, and vaginal concentration data for clindamycin phosphate are presented in FIG. 7. Vaginal clindamycin phosphate concentrations were not quantifiable in 3 of the subjects. Clindamycin phosphate levels, as measured on swabs, indicated that in nearly all subjects (15 of 21) the levels were relatively high 24 hours following administration of the composition (6 subjects had values greater than 400 micrograms/g, and 9 had values between 100 and 400 micrograms/g). The levels dropped off in most participants to below the limit of quantitation 2 days following dosing. In a few participants, levels remained elevated for several days. Maximal amounts of vaginal clindamycin occurred on Day 2 with a mean value of 30.3 micrograms. The mean amount decreased by approximately 50% on Day 3.

In FIG. 7, BLQ is Below the Lower Limit of Quantification; CV % is Coefficient of Variation; Geometric CV % is Geometric Coefficient of Variation; NC is Not Calculable; ND is Not Detectable; and SD is Standard Deviation. Concentration BLQ [<2.60 (ppm)] values and ND were set to zero for calculation of amount and subsequent summary statistics. n=number of subjects in the respective category. Total presents sum of clindamycin amount from Day 2 through Day 7.

### Example 11

In this example, a manufacturing process is outlined describing air removal from the composition, as well as filing the composition into vaginal applicators is provided.

During mixing, 2000 g of benzyl alcohol and 33.0 kg of poloxamer 407 were added. Cooling and mixing continued until the solution was clear and the poloxamer was visually dissolved. The batch temperature reached approximately 11° C. during this step. This step created foam on the surface of the batch and the mixing speed was decreased to allow the foam to dissipate. Mixing speed was then increased, eventually reaching 1200 rpm for addition of the 4.00 kg of xanthan gum. After the addition of the xanthan gum, the product rapidly builds viscosity and the original mixing blade size proved inadequate. A switch was made to a 12-inch blade to better facilitate mixing at this stage of the process.

After this step in the process, the product generated a large amount of foaming. Initially, the product was transferred into a cooling, jacketed vacuum vessel and recirculated the product for several passes to help remove the air. A second approach used a Versator, which is a device especially designed to remove air from liquids and semi-solids. The product was passed through the Versator several times until all air was removed. Measurements of the viscosity after Versator treatment rose to over 3M Cpu from 1.2 Cpu at room temperature prior to using it.

Following this step, the product can be manually or filled using an automated filling machine. In some embodiments, the automated filling machine (e.g., a Capmatic automated filing machine) is especially designed to fill vaginal applicator-based products. As the viscosity of the product decreased at refrigerated temperatures, e.g., at temperatures of about 4° C. (e.g., a reduction in viscosity of 2-3×) provides a product suitable for automated filling into a vaginal applicator. In some embodiments, a cooled, jacketed vessel, feeding cooled product into the filler hopper to fill the applicators is used.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

When ranges are given by specifying the lower end of a range separately from the upper end of the range, it will be understood that the range can be defined by selectively combining any one of the lower end variables with any one of the upper end variables that is mathematically possible.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method for treating bacterial vaginosis, comprising:
applying a composition to a vagina of a subject, the composition comprising a poloxamer, a stabilization polymer, and an active ingredient for treating bacterial vaginosis, the composition having a viscosity of at least 1.5 million cP when applied to the vagina of the subject.

2. The method of claim 1, wherein the subject exhibits one or more of the following symptoms due to bacterial vaginosis:
(i) vaginal odor;
(ii) vaginal discharge;
(iii) recurrent bacterial vaginosis.

3. The method of claim 1, wherein the subject has bacterial vaginosis or is at risk of bacterial vaginosis.

4. (canceled)

5. The method of claim 1, wherein the subject is human.

6. The method of claim 1, wherein the composition is a gel.

7. The method of claim 1, wherein the poloxamer comprises poloxamer 407.

8. The method of claim 1, wherein the stabilization polymer comprises xanthan gum.

9. The method of claim 1, wherein the composition comprises air at no more than 15 vol %.

10. The method of claim 1, comprising applying the composition through an applicator to the vagina.

11. The method of claim 1, comprising applying the composition as a single dose.

12. The method of claim 1, comprising applying the composition to the subject only once.

13. The method of claim 1, comprising applying the composition to the subject no more than once every 30 days.

14. The method of claim 2, wherein the composition causes a decrease of at least 50 vol % in vaginal discharge over a period of at least a week, relative to an equal period before application of the composition.

15. The method of claim 1, wherein the active ingredient for treating bacterial vaginosis comprises clindamycin and/or a salt thereof.

16-34. (canceled)

35. The method of claim 15, wherein the composition releases the clindamycin and/or salt thereof at a rate such that the vagina has a mucosal concentration of the clindamycin and/or salt thereof of at least 100 micrograms/g.

36-47. (canceled)

48. A composition, comprising:
a poloxamer;
a stabilization polymer; and
clindamycin and/or a salt thereof,
wherein:
(i) the composition, when exposed to a vagina of a human subject, releases clindamycin such that after 72 hours, the subject has a blood concentration of at least 5 ng/mL of clindamycin;
(ii) when 5 g of the composition is immersed in 0.5 ml of saline solution having a pH 4.6, the composition releases sufficient clindamycin into the saline solution to maintain a concentration of at least 1.4 micrograms/ml after 72 hours; or
(iii) wherein the composition has a viscosity at room temperature of at least 1.5 million cP.

49-100. (canceled)

101. A method of making a composition, comprising:
providing a composition comprising a poloxamer, a stabilization polymer, and an active ingredient for treating bacterial vaginosis; and
(i) removing air from the composition; or
(II) exposing the composition to a pressure of less than 100 mbar (absolute) for at least 30 minutes to form a gel.

102-119. (canceled)

120. A composition, comprising:
a poloxamer;
a stabilization polymer; and
an active ingredient for treating bacterial vaginosis,
wherein the composition is made by a process of claim 101.

121-140. (canceled)

141. The composition of claim 48 for treatment of bacterial vaginosis, wherein the composition comprises each of the following:
poloxamer 407 at 10-20 wt %;
xanthan gum at 1-5 wt %;
clindamycin and/or a salt thereof at 1-5 wt %;
citrate and/or a citrate salt at 0.5-2 wt %;
benzyl alcohol at 0.5-5 wt %; and
water at 60-90 wt %.

142-176. (canceled)

177. A kit comprising the composition of claim 48, wherein the kit includes an applicator suitable for vaginal application.

178-181. (canceled)
